(19) United States
(12) Patent Application Publication
(10) Pub. No.: US 2002/0115894 A1
(43) Pub. Date: Aug. 22, 2002

(54) Process for the catalytic direct oxidation of unsaturated hydrocarbons in the gas phase

(76) Inventors: Markus Weisbeck, Koln (DE); Gerhard Wegener, Mettmann (DE); Herbert Dilcher, Rangsdorf (DE); Ulrich Schulke, Berlin (DE); Bernhard Lucke, Berlin (DE)

Correspondence Address: BAYER CORPORATION, PATENT DEPARTMENT, 100 BAYER ROAD, PITTSBURGH, PA, 15205, US

(21) Appl. No.: 09/970,414

(22) Filed: Oct. 2, 2001

(30) Foreign Application Priority Data
Oct. 5, 2000 (DE) 10049 625.3

(51) Int. Cl. C07C 27/00

(52) U.S. Cl. 568/959

(57) ABSTRACT

The invention relates to a catalytic gas phase process for the preparation of epoxides from unsaturated hydrocarbons by oxidation with molecular oxygen in the presence of carbon monoxide and nanoscale gold particles.

### TECHNICAL FIELD OF THE INVENTION

The present invention relates to a catalytic gas phase process for the preparation of epoxides from unsaturated hydrocarbons by oxidation with molecular oxygen in the presence of carbon monoxide and nanoscale gold particles.

### BACKGROUND OF THE INVENTION

Generally speaking, direct oxidation reactions of unsaturated hydrocarbons with molecular oxygen in the gas phase—even in the presence of catalysts—do not take place at temperatures below 200° C. It is therefore difficult to prepare oxidation-sensitive oxidation products such as epoxides, alcohols or aldehydes because the secondary reactions of these products often take place more quickly than the oxidation of the olefins used.

Propene oxide is an important basic chemical. More than 60% of propene oxide is used in the plastics industry, particularly for the preparation of polyether polyols for the synthesis of polyurethanes. Additionally, propene oxide derivatives account for an even greater market share in the glycol sector, particularly for lubricants and anti-freeze.

Halogen-free commercial oxidation processes use organic compounds to transfer oxygen to propene. This indirect epoxidation occurs when organic hydroperoxides and percarboxylic acids in the liquid phase transfer their peroxide oxygens to olefins, thereby forming epoxides. Hydroperoxides are produced from the corresponding hydrocarbon by autoxidation with air or molecular oxygen. Hydroperoxides are converted to alcohols, while peroxycarboxylic acids are converted to acids. One disadvantage of indirect oxidation is the economic dependence of the propene oxide value on the market value of the secondary product as well as the cost-intensive production of the oxidizing agents.

EPO 0230949 and U.S. Pat. Nos. 4,410,501 and 4,701,428 disclose using titanium silicalites as catalysts to oxidize propene with hydrogen peroxide in the liquid phase under very mild reaction conditions to propene oxide with selectivities greater than 90%. JP 92/3 5277 1 discloses a process for achieving propene oxidation in a low yield in the liquid phase on platinum metal-containing titanium silicalites with a gas mixture composed of molecular oxygen and molecular hydrogen.

U.S. Pat. No. 5,623,090 discloses a gas phase direct oxidation of propene to propene oxide with 100% selectivity. The process described therein is a catalytic gas phase oxidation with molecular oxygen in the presence of a hydrogen reducing agent. The catalyst used is commercial titanium dioxide which is coated with nanoscale gold particles. The term nanoscale gold particles refers to gold particles with a diameter in the nanometer (nm) range. The propene conversion and the propene oxide yield are given as maximum 2.3%. However, the Au/TiO₂ catalysts achieve the approximately 2% propene conversion only for a very short time. For example, propene oxide yield falls by 50% after only about 2 hours at moderate temperatures (40° C.-50° C.). See Haruta et al., 3rd World Congress on Oxidation Catalysis, 1997, p. 965-970, FIG. 6. The disadvantage of this process is, therefore, that the epoxide yield is not only low, but is also greatly reduced even further by rapid deactivation.

DE 198 04 709 and DE 198 04 712 both disclose a process wherein the propene yield could be increased to greater than 5% and the catalyst life increased to several days by using catalysts which are produced from titanium oxide hydrates coated with nanoscale gold particles. However, the catalytic direct oxidation reactions are carried out in the presence of hydrogen, not carbon monoxide.

Catalysts containing gold and titanium are known. See U.S. Pat. No. 5,623,090; WO-98/00415; WO-98/00414; WO-99/43431; EPO 0827779; and DE 199 18 431. However, the catalyst systems described in the foregoing patents involve systems in which nanoscale gold particles are applied to titanium dioxide-silica mixed oxides. Additionally, these patents disclose that direct oxidation reactions are carried out in the presence of hydrogen and not carbon monoxide. Furthermore, the patents disclose that the direct oxidation reactions are not carried out at temperatures less than 30° C.

EPO 0916403 discloses a process for gas phase direct oxidation wherein carbon monoxide instead of hydrogen is used as the reducing agent. The catalysts described in this patent are based on silica which is coated with titanium dioxide and nanoscale gold particles. However, the process disclosed in EPO 0916403 indicates a maximum propene conversion of 0.39% with a propene oxide selectivity of 27% at temperatures of 30° C.-300° C.

DE 198 47629 discloses a catalytic gas phase direct oxidation process in the presence of hydrogen, oxygen and carbon monoxide. The catalysts disclosed in this reference are based on silica coated with titanium dioxide and nanoscale gold particles. However, the direct oxidation reaction disclosed in this patent is not carried out at temperatures greater than 30° C.

For the foregoing reasons, it would be desirable to develop an improved catalyst with markedly better initial activity and with greatly increased catalytic life for use in gas phase direct oxidation. Additionally, it would be desirable to develop an improved catalyst which can be used in gas phase direct oxidation at low temperatures.

### SUMMARY OF THE INVENTION

The invention relates to a process for the oxidation of unsaturated hydrocarbons with molecular oxygen in the gas phase in the presence of carbon monoxide and titanium oxide hydrate coated with nanoscale gold particles.

### BRIEF DESCRIPTION OF THE DRAWING

FIG. 1 shows a plot of propene oxide consumption versus time during propene oxidation with CO/O₂ mixtures in accordance with the present invention.

### DETAILED DESCRIPTION OF THE INVENTION

Analysis by X-ray absorption spectroscopy indicates that in the catalytically active state, the gold is present chiefly in the metallic state. Small proportions of gold may also be present in a higher oxidation state. Transient Electron Microscopy (“TEM”) photos indicate that the greatest proportion of the gold present is on the surface of the support material. The gold is in the form of gold clusters on the nanometer scale. The gold forms particles (clusters) which have an average diameter of less than 4 nm.

The nanoscale gold particles are immobilized in an adherent manner on the surface of the support. The amount of gold applied to the support will vary according to the surface, the pore structure and/or the chemical nature of the surface of the support. The properties of the support play an important part in the catalytic effect. Preferably, gold concentration is in the range from about 0.005 to 4 wt. %, based on the total weight of the catalyst composition, preferably from about 0.01 to 2 wt. %, based on the total weight of the catalyst composition, and, most preferably, from about 0.02 to 1.5 wt. %, based on the total weight of the catalyst composition. Gold concentrations higher than these ranges do not bring about an increase in catalytic activity. For economic reasons, the noble metal content is the minimum amount required to obtain the highest catalyst activity.

Any crystal structure of the material based on titanium oxide hydrate can be used in the present invention. Amorphous and anatase modifications are preferred structures. It is often advantageous if the titanium oxide hydrate is present not as a pure component but as a complex material, such as in combination with other oxides, particularly silicon. The surface should be at least about 1 m²/g, preferably in the range from about 25 m²/g to 700 m²/g, measured according to DIN 66 131.

The catalysts for the process of the invention are preferably prepared by the “deposition-precipitation” method. In this method, an aqueous solution of an inorganic or organic gold compound is added dropwise to a stirred aqueous suspension of the titanium oxide hydrate used as the catalyst support. Preferably, a water-containing solvent is used. However, other solvents such as alcohol may also be used.

If bases such as sodium carbonate or alkali or alkaline earth liquor up to a pH of about 7 to 8.5 are added to titanium oxide hydrate suspensions containing tetrachloro-auric acid, gold is precipitated in the form of Au(III)chlorohydroxo or oxohydroxo complexes, or as gold hydroxide on the titanium oxide hydrate surface. In order to bring about a uniform deposition of nanoscale gold particles, the change in the pH must be controlled by a slow dropwise addition of this aqueous alkaline solution.

As the deposited gold compounds dissolve in the excess of alkali liquor with the formation of aurates [Au(OH)₄]⁻ or AuO₂⁻, the pH is adjusted to 7 to 8.5. In order to prevent higher pH values from occurring at the site of the dropwise addition, the aqueous alkaline solutions are added by means of an impeller shaft or agitator blades.

Precipitated gold (III) hydroxide cannot be isolated as such but rather it is converted during drying to the metahydroxide AuO(OH) or Au₂O₃, which decomposes to elemental gold with the release of oxygen during calcination at temperatures above 150° C.

Amorphous, surface-rich hydrated titanium oxide hydrates coated with gold have improved catalytic activities during epoxidation of propene to propene oxide. The hydrated titanium oxides useful in the invention have a water content of from about 5 to about 50 wt. %, based on the total weight of titanium oxide hydrate, and surfaces greater than 50 m²/g. Initial propene oxide yields of greater than about 2.4% are obtained with a catalyst containing about 0.5 wt. % of gold, based on the total weight of the catalyst composition.

The water content of the titanium oxide hydrates useful in the present invention is usually from about 5 to 50 wt. %, based on the total weight of the titanium oxide hydrate, preferably from about 7 to about 20 wt. %, based on the total weight of the titanium oxide hydrate. In a preferred process of the present invention, gold is applied to titanium oxide hydrate in a precipitation step in the form of Au(III) compounds to form a suspension. The suspension then undergoes calcination in a stream of air at 350° C. to 500° C. to form an active catalyst material from the suspension.

Low sulfate contents in the TiO(OH)₂ preliminary steps often brings about an improvement in the properties of the catalysts. Hence, in the invention it is preferable to use catalysts based on titanium oxide hydrate having a sulfate content from about 0.00 to about 6 wt. %, based on the total weight of the titanium oxide hydrate, preferably about 0.1 to about 1 wt. % based on the total weight of the titanium oxide hydrate.

In the process according to the invention, propene oxide yields on the active gold titanium oxide catalysts do not decrease during the oxidation of propene with molecular oxygen in the presence of carbon monoxide, but rather remain constant over a period of time. Another advantage of the process of the invention is that the reaction may also be carried out at temperatures below 30° C. At these low temperatures, the oxidation reaction takes place in a particularly selective manner.

The process of the invention may be used for all hydrocarbons. “Hydrocarbon” is defined as unsaturated or saturated hydrocarbons such as olefins or alkanes which may also contain heteroatoms such as N, O, P, S or halogens.

The organic component to be oxidized may be acyclic, monocylic, bicyclic or polycyclic and may be monoolefinic, diolefinic or polyolefinic. In the case of organic components having two or more double bonds, the double bonds may be conjugated and non conjugated. It is preferable to oxidize hydrocarbons which form oxidation products having a partial pressure such that the product can be removed constantly from the catalyst. Preferred hydrocarbons are unsaturated and saturated hydrocarbons having 2 to 20, preferably 2 to 10 carbon atoms, particularly ethene, ethane, propene, propane, isobutane, isobutylene, but-1-ene, but-2-ene, cis-but-2-ene, trans-but-2-ene, buta-1,3-diene, pentene, pentane, hex-1-ene, hex-1-ane, hexadiene, cyclohexene, benzene.

The catalysts may be used in any physical form for oxidation reactions. Such forms include, but are not limited to, ground powders, spherical particles, pellets, and extrudates.

The relative molar ratio of hydrocarbon, oxygen, carbon monoxide and, optionally, a diluent gas, may vary widely. The starting product ratios of hydrocarbon to molecular oxygen are preferably greater than 1 and of carbon monoxide to molecular oxygen preferably greater than 2.

The molar amount of hydrocarbon used in relation to the total number of moles of hydrocarbon, oxygen, carbon monoxide and diluent gas may vary widely. An excess of hydrocarbon, based on oxygen, is preferably used (on a molar basis). The hydrocarbon content is typically greater than 1 mole %, based on the total moles of reaction gas mixture, and less than 60 mole %, based on the total moles of reaction gas mixture. Hydrocarbon contents used are preferably in the range from 5 to 15 mole %, based on the total moles of reaction gas mixture, more preferably in the range from 15 to 35 mole %, based on the total moles of reaction gas mixture. As the hydrocarbon content increases, the productivity rises.

Oxygen may be used in various forms. Such forms include, but are not limited to, purified oxygen, air and nitrogen oxide. Molecular oxygen is preferred. The molar proportion of oxygen in relation to the total number of moles of hydrocarbon, oxygen, carbon monoxide and diluent gas, may vary widely. The oxygen is used preferably in a deficient molar amount with respect to that of the hydrocarbon, preferably from 1 to 6 mole % of oxygen, based on the total moles of reaction gas mixture, more preferably 6 to 15 mole % of oxygen, based on the total moles of reaction gas mixture. As the oxygen contents increase, productivity rises. However, for safety reasons, an oxygen content of less than 20 mole %, based on the total moles of reaction gas mixture, should be selected.

The molar proportion of carbon monoxide in relation to the total number of moles of hydrocarbon, oxygen, carbon monoxide and optionally diluent gas may vary widely. The carbon monoxide may be used in various forms. Those forms include, but are not limited to, purified carbon monoxide or synthesis gas. Typical carbon monoxide contents are greater than 0.1 mole %, based on the total moles of reaction gas mixture, preferably 5 to 80 mole %, based on the total moles of reaction gas mixture, more preferably 10 to 65 mole %, based on the total moles of reaction gas mixture.

Optionally, a diluent gas such as nitrogen, helium, argon, methane, carbon dioxide or the like, preferably gases having an inert behavior, may be added to the starting gases. Mixtures of the inert components described may also be used. The addition of inert components is favorable for the transport of the heat liberated from this exothermic oxidation reaction and favorable from a safety point of view.

Preferably, gaseous diluent components such as nitrogen, helium, argon, methane and, optionally, water vapor and carbon dioxide are used. Water vapor and carbon dioxide are not completely inert but in very small concentrations, i.e., less than 2 vol. %, they bring about a positive effect.

The reaction temperature of the process of the invention is below 30° C., preferably in the range from 0° C. to 30° C., more preferably in the range from 15° C. to 30° C.

### EXAMPLES

The following examples further illustrate details for the process according to the invention:

### Example 1

100 g of titanium oxide hydrate (BET surface, i.e., the surface determined in accordance with the method described in Brunauer, Emmett and Teller, J. Am. Chem. Soc., Volume 60, page 309 (1938), 320 to 380 m²/g, 10 to 14% water, 0.2% sulfate) were introduced, with stirring, into a solution of 1.0 g of tetrachloro-auric acid trihydrate, HAuCl₄0.3H₂O in 2000 ml of distilled water, and the suspension was adjusted immediately to a pH of 7.8 to 8.0 with 0.1 N NaOH. While maintaining a constant pH and stirring vigorously, the suspension was then heated to 333° K to 343° K. within a period of 30 min by adding 0.1 N NaOH, and kept at this temperature for 1 h. The consumption of 0.1 N NaOH was 265 ml.

The occurrence of greatly increased hydroxide ion concentrations at the point of introduction into the suspension was prevented by introducing the 0.1 N NaOH by way of an impeller shaft and agitator blades.

The colorless suspension was cooled to 313° K. within a period of 75 min and a solution, adjusted to pH 8, of 4.6 g of magnesium citrate MgHCitr.5H₂O in 300 ml of distilled water was added, with stirring, and stirring was continued for 1 h. The solid was then separated by centrifugation and washed 3 times with 1800 ml of distilled water in each case. The wash process was intensified by dispersing the solid particles in the wash water with a stirrer operating at high speed (20,000 rpm).

The damp precursor thus prepared was dried for 16 h at 303° K. at 8 mbar and for 1 h at 423° K. at 1 bar, and then heated to 673° K. at a rate of heating of 2° K., and kept at this temperature for 2 h.

The gray—blue—purple colored catalyst had a gold content of 0.5%. The BET surface was 110 m²/g. The particle diameter of the gold clusters, determined by TEM measurements, was 1.5 nm on average. The Au clusters had well developed 111 faces and were anchored on the surface of the titanium dioxide support. 111 faces refers to the Miller indices obtained for the face.

The results of the catalytic epoxidation of propene are summarized in Table 1.

### Example 2

The catalyst was prepared in a similar way to Example 1 but the tetrachloro-auric acid solution was introduced dropwise into the reaction solution from a dropping funnel and the damp precursor was dried at 373° K. at 1 bar and tempered for 4 h at 673° K.

The gray—blue—purple colored catalyst had a gold content of 0.5%. The BET surface was 105 m²/g. The particle diameter of the gold clusters, determined by TEM measurements, was 4 nm on average. The 111 faces of the Au clusters were considerably disturbed.

### Example 3

The gas phase direct oxidation was examined in a fixed bed tubular reactor (diameter 1 cm, length 20 cm) made of double-walled glass, which was temperature controlled by means of a thermostat. A static mixing and temperature control section was installed upstream of the reactor. The gold supported catalyst was placed on a glass frit. The catalyst loading was 1.1 l/g cat·h⁻¹.

The starting gases were introduced into the reactor in a downward direction by means of a mass flow controller.

The starting gas ratios ranged from O₂/CO/H₂/C₃H₆/Ar:0.1/0.175/0.025/0.1/0.7 to 0.1/0.1/0.1/0.1/0.7.

The reaction temperature was from 10° C. to 50° C.

The reaction gas mixture was analyzed by means of gas chromatography with a flame ionization detector (“FID”) (all organic compounds), a methanizer (organic compounds, CO and CO₂) and a thermo-coupled detector (“TCD”) (permanent gases, CO, CO₂, H₂O). The plant was controlled by means of a central data acquisition system.

TABLE 1
Results of direct oxidation of propene to
propene oxide in the presence of CO

| | Reaction | Propene oxide | Propene oxide |
|---|---|---|---|
| | temperature | selectivity | yield |
| Catalyst | (° C.) | (%) | (%) |
| Example 1 | 10 | >99 | 1.4 |
| | 15 | >99 | 2.1 |
| | 20 | >99 | 2.4 |
| | 50 | 90-95 | 1.6 |
| Example 2 | 15 | 0 | 0 |

The comparison of the catalysts prepared according to Examples 1 and 2 indicates that only catalysts comprising gold particles having an average diameter of less than 4 nm are catalytically active.

### Example 4

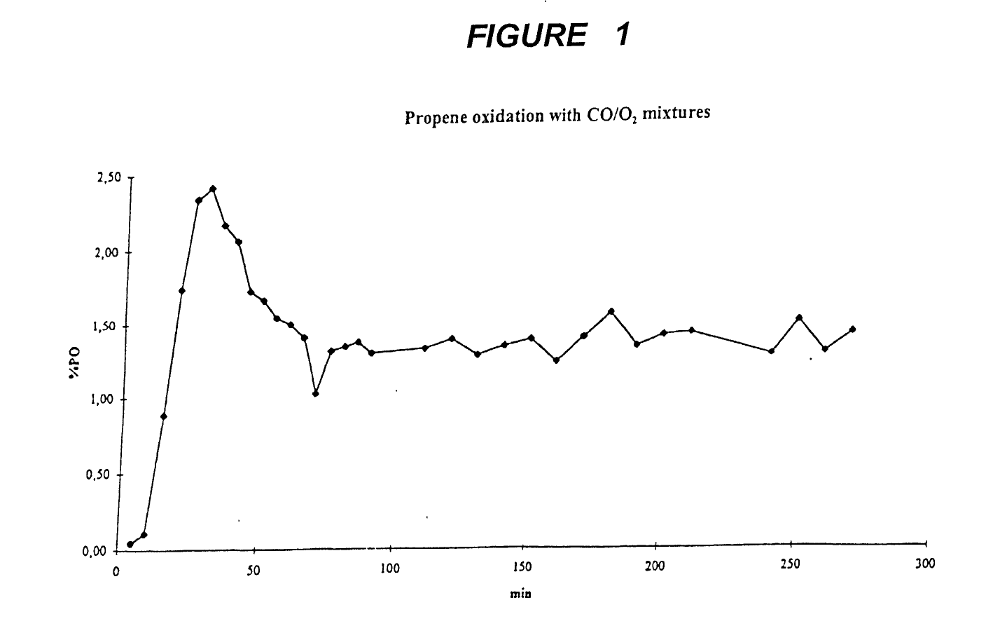

The preparation of the catalyst and oxidation took place as described in Example 1. The oxidation of propene was monitored over a period of 6 h under the same reaction conditions as in Example 3 and the propene oxide yield was determined at regular intervals. After a brief yield peak, the yield remained constant at a value of 1.4% during the observation period of a total of 6 h (see FIG. 1).

The ratio was propene:CO:O₂:Ar=1:2:1:7, the temperature was 10° C.

### Comparison Example 5

In a comparison test with the same catalyst according to Example 1, it could be shown with a working gas of H₂:propene:O₂=7.5:2:0.5 under otherwise identical conditions that, after a brief yield peak of over 2%, a continuous fall in activity became apparent after only 2 h. The yield fell to less than 0.5% after only 4.5 h.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the oxidation of unsaturated hydrocarbons comprising contacting a catalyst composition comprising (i) titanium oxide hydrate, and (ii) gold with a reaction gas mixture comprising (i) hydrocarbon, (ii) oxygen, (iii) carbon monoxide, and, optionally, (iv) a diluent gas, wherein the gold forms particles that have an average diameter of less than 4 nm.

2. The process according to claim 1, wherein the oxidation process is conducted at temperatures below 30° C.

3. The process according to claim 1, wherein the oxidation process is conducted at temperatures in the range of from 0° C. to 30° C.

4. The process according to claim 1, wherein the oxidation process is conducted at temperatures in the range of from 15° C. to 30° C.

5. The process according to claim 1 wherein the titanium oxide is anatase.

6. The process according to claim 1, wherein the titanium oxide hydrate is amorphous.

7. The process according to claim 1, wherein the titanium oxide hydrate is a complex material.

8. The process according to claim 7, wherein the complex material comprises titanium oxide hydrate and silicon or silica.

9. The process according to claim 8, wherein the titanium oxide hydrate has a sulfate content of from about 0.00 to 6 wt. %, based on the total weight of the titanium oxide hydrate.

10. The process according to claim 8, wherein the titanium oxide hydrate has a sulfate content of from about 0.1 to 1 wt. %, based on the total weight of the titanium oxide hydrate.

11. The process according to claim 1, wherein the surface of the titanium oxide hydrate is at least 1 m²/g.

12. The process according to claim 1, wherein the surface of the titanium oxide hydrate is at least 25 m²/g.

13. The process according to claim 1, wherein the surface of the titanium oxide hydrate is in the range of from about 25 m²/g to about 700 m²/g.

14. The process according to claim 1, wherein the titanium oxide hydrate has a water content of from about 5 to about 50 wt. %, based on the total weight of the titanium oxide hydrate.

15. The process according to claim 1, wherein the titanium oxide hydrate has a water content of from about 7 to 20 wt. %, based on the total weight of the titanium oxide hydrate.

16. The process according to claim 1, wherein the gold is metallic.

17. The process according to claim 1, wherein the gold is applied to the titanium oxide hydrate.

18. The process according to claim 17, wherein the concentration of gold applied to the titanium oxide hydrate is in the range of from about 0.005 to 4 wt. %, based on the total weight of the catalyst composition.

19. The process according to claim 17, wherein the concentration of gold applied to the titanium oxide hydrate is in the range of from about 0.01 to 2 wt. %, based on the total weight of the catalyst composition.

20. The process according to claim 17, wherein the concentration of gold applied to the titanium oxide hydrate is in the range of from about 0.02 to 1.5 wt. %, based on the total weight of the catalyst composition.

21. The process according to claim 17, wherein the gold is applied to the surface of the titanium oxide hydrate.

22. The process according to claim 21, wherein the gold is substantially immobilized on the surface of the titanium oxide hydrate.

23. The process according to claim 1, wherein the catalyst composition is calcined in a stream of air at 350° to 500° C.

24. The process according to claim 1, wherein the hydrocarbon is an olefin or alkane.

25. The process according to claim 1, wherein the hydrocarbon and oxygen are present in the reaction gas mixture in a ratio of greater than one.

26. The process according to claim 1, wherein the carbon monoxide and oxygen are present in the reaction gas mixture in a ratio greater than two.

27. The process according to claim 1, wherein hydrogen is present in the reaction gas mixture in a range of from about greater than about 1 mole % to about less than 60 mole %, based on the total moles of reaction gas mixture.

28. The process according to claim 1, wherein hydrogen is present in the reaction gas mixture in a range of from about 5 mole % to about 15 mole %, based on the total moles of reaction gas mixture.

29. The process according to claim 1, wherein hydrogen is present in the reaction gas mixture in a range of from about 15 mole % to about 35 mole %, based on the total moles of reaction gas mixture.

30. The process according to claim 1, wherein oxygen is in the form of molecular oxygen.

31. The process according to claim 1, wherein oxygen is present in the reaction gas mixture in a range of from about 1 mole % to about 6 mole %, based on the total moles of reaction gas mixture.

32. The process according to claim 1, wherein oxygen is present in the reaction gas mixture in a range of from about 6 mole % to about 15 mole %, based on the total moles of reaction gas mixture.

33. The process according to claim 1, wherein carbon monoxide is in the form of purified carbon monoxide or synthesis gas.

34. The process according to claim 1, wherein carbon monoxide is present in the reaction gas mixture in a range of from about greater than 0.1 mole % to about 80 mole %, based on the total moles of reaction gas mixture.

35. The process according to claim 1, wherein carbon monoxide is present in the reaction gas mixture in a range of from about 5 mole % to about 80 mole %, based on the total moles of reaction gas mixture.

36. The process according to claim 1, wherein carbon monoxide is present in the reaction gas mixture in a range of from about 10 mole % to about 65 mole %, based on the total moles of reaction gas mixture.
